(19) United States
(12) Patent Application Publication
(10) Pub. No.: US 2008/0269306 A1
(43) Pub. Date: Oct. 30, 2008

(54) Antidepressant Medicament Comprising Idazoxan and a Selective Serotonin Reuptake Inhibitor

(75) Inventors: Torgny Svensson, Lidingo (SE); Salomon Z. Langer, London (GB)

Correspondence Address: BACON & THOMAS, PLLC, 625 SLATERS LANE, FOURTH FLOOR, ALEXANDRIA, VA, 22314-1176, US

(73) Assignee: ALPHA 2 PHARMACEUTICA AB, STOCKHOLM (SE)

(21) Appl. No.: 11/793,200

(22) PCT Filed: Dec. 27, 2005
(86) PCT No.: PCT/SE05/02045

§ 371 Date: Jun. 26, 2008

Related U.S. Application Data
(60) Provisional application No. 60/638,384, filed on Dec. 27, 2004.

(51) Int. Cl. A61K 31/4178; A61P 25/00

(52) U.S. Cl. 514/385

(57) ABSTRACT

Use of Idazoxan or an active enantiomer thereof, and treatment of patients receiving therapy with selective serotonin uptake inhibitor(s), such as Fluoxetine, Citalopram, Paroxetine, Sertraline and Fluvoxamine, are described. The patients being treated are in particular those who are suffering from severe depression, anxiety-depression, PTSD (Post Traumatic Stress Disorder), or ADHD (Attention Deficit Hyperactive Disorder).

The present invention relates to therapies that involve the administration of racemic or enantiopure α₂ adrenergic receptor antagonist Idazoxan in combination with a selective inhibitor of serotonin uptake to produce an improved antidepressant response particularly in treatment resistant depressed patients as well as in patients with other mental illnesses. Slow release formulations will be preferred for the administration of a medicament comprising Idazoxan in either racemic or the enantiopure form.

### BACKGROUND OF THE INVENTION

Depression is a serious disease, which affects 8% of the adult population. It is the most common of the mental illnesses and it also involves the possibility of a fatal outcome when it leads to suicide. Despite the availability of a considerable number of antidepressant drugs of the first and second generation, two major unmet medical needs remain without any satisfactory solution (excluding electroconvulsive therapy): the issue of drug resistance or non responders and the latency period in the onset of action of antidepressant effects. In the case of the latency period, about three to four weeks must pass before the therapeutic antidepressant effects of the drug are significant. In contrast, all the side effects of the administered antidepressant are present from the first day of drug administration. This latency period involves an enhanced danger for suicide attempts. Concerning the non responders to treatment, their size is as high as 30% of the depressed patients and it remains a priority objective. The great majority of conventional antidepressant drugs increase the brain concentrations of two neurotransmitters noradrenaline (NA) and serotonin (5-HT) or both. This effect is obtained within 24 hours of drug administration and yet, the therapeutic effect is not developed before two to three weeks of daily administration. Furthermore, in 30% of the patients no therapeutic effect is manifested and this population of patients is referred to as treatment-resistant depressed patients or patients with severe depression.

### DESCRIPTION OF THE INVENTION

The present invention refers to improved antidepressant therapeutic regimens resulting in: a) improved antidepressant efficacy with high percentage of responders, and b) rapid onset of antidepressant action. More precisely, the invention is concerned with administration of a centrally acting, potent alpha-2 adrenoceptor antagonist Idazoxan or an active enantiomer thereof to patients receiving therapy with an 5-HT uptake inhibitor. The invention is particularly concerned with the administration of Idazoxan as well as its active enantiomer as “add on” therapy in the therapy of treatment-resistant depression. The development of a slow release formulation for Idazoxan and its active enantiomer will improve the therapeutic efficacy and reduce cardiovascular side effects by enabling a once a day administration, stable therapeutic plasma levels, avoiding peaks of plasma levels which may reach circulating concentrations inducing cardiovascular side effects.

One aspect of the invention is directed to the use of 2-(1,4-benzodioxan-2-yl)-2-imidazoline (Idazoxan) or an active enantiomer thereof for the manufacture of a medicament for the treatment of a patient receiving therapy with selective serotonin uptake inhibitor(s).

In a preferred embodiment the use of the medicament is for treatment of patients suffering from severe depression, anxiety-depression, PTSD (Post Traumatic Stress Disorder), or ADHD (Attention Deficit Hyperactive Disorder) who receiving therapy with selective serotonin uptake inhibitor(s), especially patients exhibiting poor response to monotherapy with the selective serotonin uptake inhibitor(s).

In another embodiment of the invention the medicament comprises both Idazoxan or an active enantiomer thereof and selective serotonin uptake inhibitor(s), which is (are) preferably selected from the group consisting of Fluoxetine, Citalopram, Paroxetine, Sertraline and Fluvoxamine.

In a presently preferred embodiment of the invention, the medicament is in the form of a slow-release preparation.

Another aspect of the invention is directed to a method of treating a patient receiving therapy with selective serotonin uptake inhibitor(s) comprising administration to said patient of an amount of 2-(1,4-benzodioxan-2-yl)-2-imidazoline (Idazoxan) or an active enantiomer thereof which is a psychologically effective amount together with the amount of administered selective serotonin uptake inhibitor(s).

In a preferred embodiment of this aspect of the invention the patient that is treated is suffering from severe depression, anxiety-depression, PTSD (Post Traumatic Stress Disorder), or ADHD (Attention Deficit Hyperactive Disorder), in particular the patient exhibits poor response to monotherapy with the selective serotonin uptake inhibitor(s).

The selective serotonin uptake inhibitor(s) and the Idazoxan or an active enantiomer thereof are preferably administered in one pharmaceutical preparation.

In a preferred embodiment, the selective serotonin uptake inhibitor(s) is (are) selected from the group consisting of Fluoxetine, Citalopram, Paroxetine, Sertraline and Fluvoxamine.

In presently preferred embodiments of the method aspect of the invention the administered Idazoxan or an active enantiomer thereof is in the form of a slow-release preparation, and the pharmaceutical preparation comprising both the selective serotonin uptake inhibitor(s) and the Idazoxan or an active enantiomer thereof is in the form of a slow-release preparation.

Thus, the present invention provides an effective method of treatment especially for patients suffering from treatment resistant depression who have not responded adequately to treatment with classical antidepressants or therapies using selective serotonin uptake inhibitor(s), by adding to the ongoing treatment, either racemic or the active enantiomer of idazoxan.

A major advantage of this drug combination is the possibility to provide a method of treatment which can address two unmet medical needs in depression, by possessing, a) rapid onset of antidepressant action, and, b) improved antidepressant efficacy with high percentage of responders.

A further advantage of the present invention is the possibility to provide a method of treatment, which does not have the severe side effects associated with the administration of existing antidepressants. This is achieved by reducing the doses of both drugs, Idazoxan (racemic or active enantiomer) and the inhibitor of neuronal uptake of 5-HT.

Yet, another advantage of the present invention is the possibility to provide pharmaceutical compositions with appropriate formulation (slow release) for Idazoxan or an active enantiomer thereof for once a day administration and to avoid peaks of plasmatic concentration in the region of cardiovascular side effects. Such a slow release formulation will improve the therapeutic efficacy and reduce cardiovascular side effects by stabilizing therapeutic plasma levels, avoiding peaks of plasma levels which may reach circulating concentrations inducing cardiovascular side effects.

Within the scope of the present invention there is provided pharmaceutical compositions comprising a combination of (i) Idazoxan or an active enantiomer thereof, (ii) 5-HT reuptake inhibitor(s), and (iii) a pharmaceutically acceptable slow release formulation, wherein the amount of ingredients (i) and (ii) is therapeutically effective against serious mental illnesses such as treatment resistant or severe depression, anxiety-depression, PTSD (post traumatic stress disorder) and ADHD (attention deficit hyperactivity disorder). It is believed that the appropriate active enantiomer of Idazoxan should be equally active or even superior that the racemic form. The combinations of racemic Idazoxan or pure active enantiomer of Idazoxan with selective serotonin uptake inhibitor antidepressants are usually combinations with the 5-HT uptake inhibitors Fluoxetine, Citalopram, Paroxetine, Sertraline, and/or Fluvoxamine

Regarding the latency period of the antidepressant effect, it is expected to be significantly shorter than with current therapy, which takes three to four weeks and the antidepressant efficacy should be superior to inhibitors of 5-HT uptake administrated in monotherapy.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 is a diagram that shows the noradrenaline output % of baseline (BL) plotted against time in the medial prefrontal cortex after administration to rats of Citalopram 5 mg/kg s.c.+saline 1 mg/kg s.c., and Citalopram 5 mg/kg s.c.+Idazoxan 1.5 mg/kg s.c

FIG. 2 shows a diagram of the overall noradrenaline output, area under the curve, 120-270 min in FIG. 1 in % of baseline (BL).

FIG. 3 is a diagram that shows the dopamine output % of baseline (BL) plotted against time in the medial prefrontal cortex after administration to rats of Citalopram 5 mg/kg s.c.+saline 1 mg/kg s.c., and Citalopram 5 mg/kg s.c.+Idazoxan 1.5 mg/kg s.c

FIG. 4 shows a diagram of the overall dopamine output, area under the curve, 120-270 min in FIG. 3 in % of baseline (BL)

### EXPERIMENTS

### Materials and Methods

### Animals

Adult male BK1:WR (Wistar) rats weighing 230-390 g were used in all experiments. Animals arrived at least five days prior to experimental use and were housed (4 per cage [Makrolon IV]) in the animal facility under standard laboratory conditions with a 12 h light/dark cycle with lights on at 6.00 a.m. All experiments were performed between 8.00 a.m. and 6.00 p.m. Food and water were available ad lib. All experiments were approved by, and conducted in accordance with, the local Animal Ethics Committee (Stockholms Norra och Södra Försöksdjursetiska Kommittéer) (permits no. N216/00, N11/00).

### Microdialysis

The probe implantation and dialysis procedure, as well as the biochemical analyses, were similar to those previously described (Hertel et al 1996, Linner et al 1999). Anesthetized male BKI:WR (Wistar) rats (B&K Universal, Sollentuna, Sweden; sodium pentobarbital, 60 mg/kg, intraperitoneal; i.p.) were implanted with dialysis probes in the medial prefrontal cortex (mPFC) [AP: +2.6; ML: −0.6; DV: −5.2], relative to bregma and dural surface (Paxinos and Watson 1998). Dialysis occurred through a semipermeable membrane (AN69 Hospal) with an active surface length of 4 mm. Dialysis experiments were conducted approximately 48 h after surgery in freely moving rats. The dialysis probe was perfused with a physiological perfusion solution (147 mM sodium chloride, 3.0 mM potassium chloride, 1.3 mM calcium chloride, 1.0 mM magnesium chloride, and 1.0 mM sodium phosphate, pH 7.4) at a rate of 2.5 μl/min set by a microinfusion pump (Harvard Apparatus, Holliston, Mass.). On-line quantification of noradrenaline (NA) or dopamine (DA) in the dialysate was accomplished by high pressure liquid chromatography coupled to electrochemical detection. The placement of the probe was later verified in slices stained with neutral red.

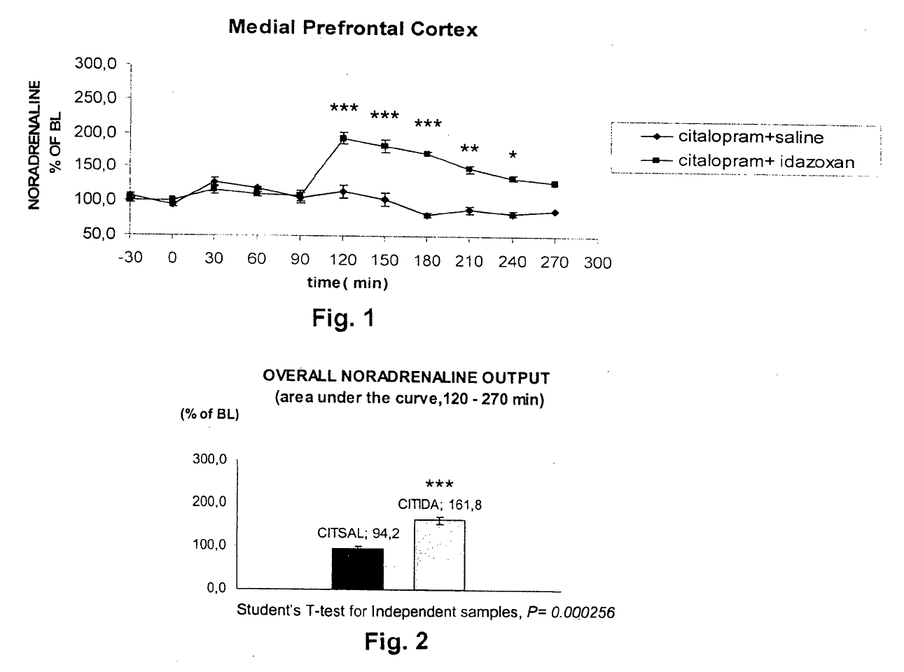

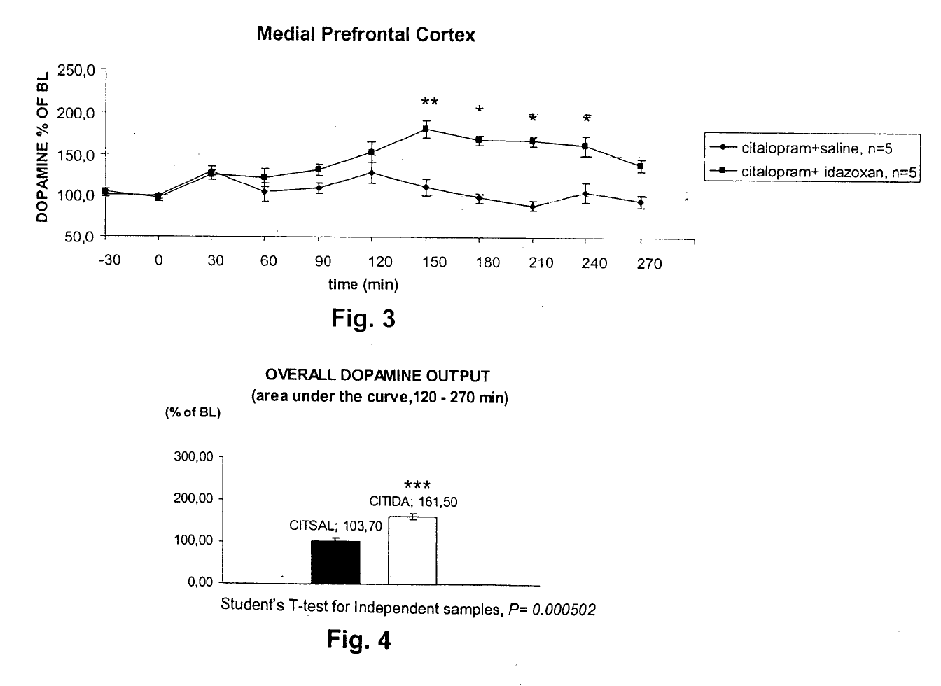

The results from the experiments are presented in the Tables 1 and 2 below and are illustrated in the FIGS. 1, 2, 3 and 4.

TABLE 1

| NORADRENALINE | | | | Medial Prefrontal Cortex | | | |
|---|---|---|---|---|---|---|---|
| TREATMENT GROUP n = 5 | | | | TREATMENT GROUP n = 5 | | | |
| Citalopram 5 mg kg s.c + | | | | Citalopram 5 mg/kg s.c + | | | |
| Saline 1 ml/kg s.c | | | | Idazoxan 1.5 mg/kg s.c | | | |
| | Mean | | | | Mean | | |
| | values | SEM | | | values | SEM | |
| | (fmol/min) | (fmol/min) | | | (fmol/min) | (fmol/min) | |
| | basal level: | | over time | | basal level: | | over time |
| | 1.081 | 0.100 | effects | | 0.829 | 0.207 | effects |
| TIME | (% Baseline) | (% Baseline) | p-value | TIME | (% Baseline) | (% Baseline) | p-value |
| −30 min | 106.5 | 2.6 | | −30 min | 100.0 | 6.2 | |
| 0 min | 93.5 | 2.6 | | 0 min | 100.0 | 6.2 | |
| CIT | | | | CIT | | | |
| 30 min | 127.8 | 5.3 | 0.129518 | 30 min | 115.7 | 8.9 | 0.579767 |
| 60 min | 118.8 | 2.6 | 0.463906 | 60 min | 109.9 | 14.3 | 0.741427 |
| 90 min | 105.5 | 8.1 | 0.746606 | 90 min | 108.0 | 6.6 | 0.742031 |
| SAL | | | | IDA | | | |
| 120 min | 114.6 | 9.1 | 0.571791 | 120 min | 193.6 | 12.3 | 0.000147 *** |
| 150 min | 103.2 | 9.6 | 0.698032 | 150 min | 182.5 | 14.1 | 0.000145 *** |
| 180 min | 81.6 | 2.9 | 0.853716 | 180 min | 171.8 | 15.4 | 0.000138 *** |
| 210 min | 88.7 | 5.1 | 0.686504 | 210 min | 148.3 | 11.8 | 0.001227 ** |
| 240 min | 83.7 | 4.1 | 0.840754 | 240 min | 135.2 | 16.6 | 0.035225 * |
| 270 min | 87.9 | 4.9 | 0.850396 | 270 min | 128.9 | 12.1 | 0.093376 |

### Statistical Analysis:

2-Way Anova (treatment×time) followed by Neuman-Keuls Post Hoc comparison test.

TABLE 2

| DOPAMINE | | | | Medial Prefrontal Cortex | | | |
|---|---|---|---|---|---|---|---|
| TREATMENT GROUP n = 5 | | | | TREATMENT GROUP n = 5 | | | |
| Citalopram 5 mg kg s.c + | | | | Citalopram 5 mg/kg s.c + | | | |
| Saline 1 ml/kg s.c | | | | Idazoxan 1.5 mg/kg s.c | | | |
| | Mean | | | | Mean | | |
| | values | SEM | | | values | SEM | |
| | (fmol/min) | (fmol/min) | | | (fmol/min) | (fmol/min) | |
| | basal level: | | over time | | basal level: | | over time |
| | 0.323 | 0.055 | effects | | 0.306 | 0.020 | effects |
| TIME | (% Baseline) | (% Baseline) | p-value | TIME | (% Baseline | (% Baseline) | p-value |
| −30 min | 101.4 | 3.5 | | −30 min | 104.1 | 3.3 | |
| 0 min | 98.6 | 3.5 | | 0 min | 95.9 | 3.3 | |
| CIT | | | | CIT | | | |
| 30 min | 129.4 | 5.7 | 0.596581 | 30 min | 125.3 | 6.9 | 0.715058 |
| 60 min | 104.5 | 11.2 | 0.714707 | 60 min | 121.2 | 12.5 | 0.778166 |
| 90 min | 109.1 | 6.4 | 0.912012 | 90 min | 131.2 | 17.4 | 0.674619 |
| SAL | | | | IDA | | | |
| 120 min | 127.8 | 12.6 | 0.601813 | 120 min | 153.4 | 12.1 | 0.085398 |
| 150 min | 110.3 | 10.6 | 0.948449 | 150 min | 180.8 | 19.3 | 0.001165 ** |
| 180 min | 97.7 | 5.7 | 0.952285 | 180 min | 168.0 | 21.4 | 0.011522 * |
| 210 min | 88.0 | 5.7 | 0.963173 | 210 min | 166.9 | 15.6 | 0.012322 * |
| 240 min | 104.5 | 12.1 | 0.927253 | 240 min | 161.8 | 17.2 | 0.026447 * |
| 270 min | 94.1 | 7.7 | 0.992125 | 270 min | 138.2 | 13.7 | 0.449311 |

These results demonstrate that under conditions of the administration of a dose of citalopram which does not per se affect noradrenaline or dopamine output in the medial prefrontal cortex of the awake freely moving rat, the addition of a low dose of Idazoxan elicits a significant increase in both noradrenaline and dopamine output, lasting approximately 2 hours. Thus, with the addition of Idazoxan an enhanced noradrenaline overflow in brain is also obtained, in analogy with the effect of selective noradrenaline uptake inhibitors. Moreover, an enhanced dopamine output is generated in the prefrontal cortex. As depression frequently is associated with an impaired cognitive functioning, and dopamine in the prefrontal cortex facilitates cognitive functions, such as working memory (see Marcus et al 2005), this effect of adding Idazoxan to selective serotonin inhibitors may significantly contribute to the therapeutic efficacy of the combined treatment.

It has earlier been discovered that receptors for neurotransmitters are not only present postsynaptically, where the mediate responses but also in the cell body and the terminals of the neuron. Since these receptors are activated by the transmitter released from the same neuron, they are referred to as “autoreceptors”. Somatodendritic autoreceptors mediate inhibition of the firing of the cell body, while terminal autoreceptors mediate inhibition of the depolarization-evoked, calcium-dependent, transmitter release. The idea that increasing the availability of 5-HT is effective in the treatment of depression is the rationale for the use of inhibitors of neuronal uptake of 5-HT in the treatment of depression according to the invention. Since neuronal uptake of 5-HT represents the main physiological mechanism for the termination of action and the inactivation of the released transmitter, inhibition of the sodium chloride dependent transporter is expected to produce an increase in the concentrations of the relevant transmitter in the synaptic cleft. Concomitantly, however, the elevated synaptic levels of the transmitter, activate inhibitory autoreceptors in the cell body and in the nerve endings leading to inhibition of transmitter release and counteracting the expected effect on inhibition of reuptake. As a function of time (and perhaps the cause of the latency period of the therapeutic effect in depression) sub-sensitivity of the presynaptic autoreceptors develops and this phenomenon leads to a progressive removal of the presynaptic inhibition of firing of the cell body and transmitter release from the nerve terminals. Accordingly, the α₂-adrenergic receptor antagonist Idazoxan or an active enantiomer thereof, by blocking the presynaptic alpha-2 receptors which inhibit transmitter release will produce within 24 hrs the desired blockage of presynaptic α₂-adrenergic receptors and therefore, as shown acutely in numerous experiments on transmitter release both in vitro and in vivo.

The present invention provides an improved treatment for the patients suffering from severe depression who have proven resistant to treatments with known inhibitors of 5-HT as therapeutic strategy.

Preferably, the α₂-adrenergic receptor antagonist utilized according to the invention is the selective antagonist Idazoxan or an active enantiomer thereof, namely [(±)-2-(1,4-benzodioxan-2-yl)-2-imidazoline] or/and the optically pure active enantiomer of Idazoxan [(+)-2-(1,4-benzodioxan-2-yl)-2-imidazoline]. i.e., a drug whose principal pharmacological effect in vitro and in vivo is antagonism of α₂-adrenergic receptors. Any additional pharmacological effects should be minor in comparison to this principal effect. Idazoxan is a highly selective α₂-adrenergic receptor antagonist in the periphery and the central nervous system (see Doxey et al., Arch. Pharmacol. 325:136-44, 1984).

In the preferred embodiment of the method according to the invention, the α₂-adrenergic receptor antagonist is administered to treatment resistant patients presently undergoing chronic antidepressant administration of 5-HT reuptake inhibitors. The two compounds also can be administered together, at the beginning of treatment or by “add on” therapy with Idazoxan as shown in the example below.

### Preferred Dosages:

(±) Idazoxan 20 or 40 mg or 80 mg 3 times a day.
(+) active enantiomer of Idazoxan 10 or 20 mg or 40 mg 3 times a day.
Slow release (±) Idazoxan 120 mg or 240 mg once a day.
Slow release (+) active Idazoxan 60 mg or 120 mg once a day.

As earlier mentioned herein, in one embodiment of the present invention, both the α₂-adrenergic receptor antagonist Idazoxan or an active enantiomer thereof and serotonin uptake inhibitors can be administrated in a single pharmaceutical preparation or composition, as a combination or by “add on” therapy, in combination with known pharmaceutically acceptable carriers (formulation such as slow release). Such pharmaceutical composition, thus constitute another aspect of the present invention.

These compositions may be prepared from conventional materials by known procedures.

Compositions, i.e. pharmaceutical preparations or medicaments, within the present invention can be adapted for oral or parenteral administration, as well as for enteral administration orally or through mucus membranes, i.e. intranasally, sublingually, buccally or rectally.

Examples of compositions for oral administration include capsules, tablets, dispersible powders, granules, syrups, elixirs and suspensions. These compositions contain one or more conventional adjuvants, such as sweetening agents, flavoring agents, coloring agents and preserving agents.

Tablets can contain the active ingredients in a mixture with conventional pharmaceutically acceptable excipients. These include inner carriers, such as calcium carbonate, sodium carbonate, lactose, and talc; granulating and disintegrating agents, such as starch and alginic acid; binding agents such as starch, gelatin acacia; and lubricating agents, such as magnesium stearate, stearic acid and talc. Tablets may be uncoated or coated by known techniques to delay disintegration and absorption in the gastrointestinal tract thereby providing a sustained action over a longer period of time (Slow release). Capsules may contain the active ingredients alone or in admixture with an inert solid carrier, such as calcium carbonate, calcium phosphate or kaolin, Similarly, suspensions, syrups and elixirs may contain the active ingredients in mixture with any of the conventional excipients utilized in the preparation of such compositions. This includes suspending agents such as methylcellulose, tragacanth and sodium alginate; wetting agents such as lecithin, polyoxyethylene stearate or polyoxyethylene sorbitan monoleate; and preservatives. The slow release formulation (similarly as for tablets) may be applied on capsules with combination of active ingredients as well.

Specific examples of embodiments of the invention comprise:

Administration of the active enantiomer (+) Idazoxan in combination with Citalopram (add on therapy) by the same protocol as administration with Citalopram and (±) Idazoxan.

Administration of the (−) Idazoxan in combination with Citalopram (add on therapy) by the same protocol as administration of Citalopram and (±) Idazoxan.

### Formulations

Pharmaceutical compositions, preparations or medicaments used in the present invention include the α₂-adrenergic receptor antagonist Idazoxan in various proportions and formulations (slow release and conventional). For example, a white round film-coated tablet can include 20 mg of (+) active enantiomer of Idazoxan together with: microcrystalline cellulose, colloidal silicon dioxide, croscarmellose sodium, talc, magnesium stearate, hydroxypropyl methylcellulose, titanium dioxide, and polyethylene glycol 400 (PEG), modified starch, polyacrylic acid (PAA), and sodium carboxymethylcellulose (CMC), modified maize starch, containing 5% (w/w) PAA and PEG with a mol. wt of 300,000 daltons.

### Chemical Structure of Idazoxan

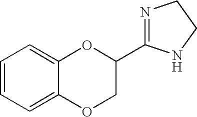

### Chemical Structure of 5-HT Uptake Inhibitors

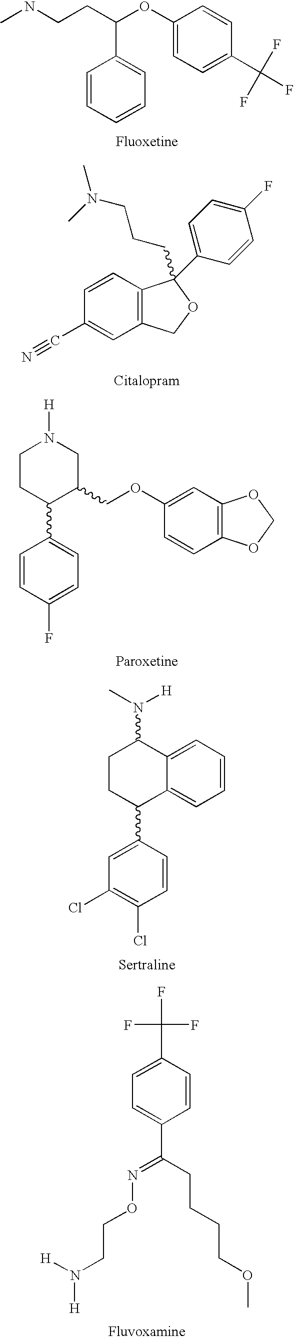

### LIST OF REFERENCES

1. Anden N E. Corrodi H. Fuxe K. Hokfelt T. Rydin C. Svensson T. “Evidence for a central noradrenaline receptor stimulation by clonidine”. Life Sciences. 9:513-23, 1970.
2. J. C. Doxey, A. G. Roach and C. F. C. Smith; “Studies on RX 781094: a selective, potent and specific antagonist of alpha-2 adrenoceptors”, Br. J. Pharmac, 78, 489-505, 1983.
3. M. L. Dubocovich, S. Z. Langer; “Negative feed-back regulation of NA release by nerve stimulation in the perfused cat's spleen: differences in potency of phenoxybenzamine in blocking the pre- and post-synaptic adrenergic receptors”, J. Physiol., 237, 505-519, 1974.
4. P. Glue, S. Wilson, G. Campling and D. Nutt; “α₂-adrenoceptor control of cortisol and ACTH in normal volunteers: preliminary open trial of the effects of acute and chronic idazoxan”, Psychoneuroendocrinology, 17, 261-266, 1992.
5. Grenhoff J. Svensson T H. “Clonidine modulates dopamine cell firing in rat ventral tegmental area”. European Journal of Pharmacology. 165:11-18, 1989.
6. F. Grossman, W. Z. Potter, E. A. Brown, G. Maislin; “A double-blind study comparing idazoxan and bupropion in bipolar depressed patients”, Journal of Affective Disorders, 56, 237-243, 1999.
7. Hertel P. Fagerquist M V. Svensson T H. “Enhanced cortical dopamine output and antipsychotic-like effects of raclopride by alpha2 adrenoceptor blockade”. Science. 286:105-7, 1999.
8. P. Hertel, G. G. Nomikos, T. H. Svensson,; “Idazoxan preferentially increases dopamine output in the rat medial prefrontal cortex at the nerve terminal level”, Euro. J. of Pharmac., 371,153-158, 1999.
9. S. Z. Langer; “Presynaptic receptors and their role in the regulation of transmitter release”, Proceedings of the Sixth Gaddum Memorial lecture, Mill Hill, Br. J. Pharmac, 60, 481-497, 1977.
10. S. Z. Langer; “Presynaptic regulation of the release of catecholamines”, Pharmac. Reviews, 32, 337-362, 1981.
11. S. Z. Langer; “25 years since the discovery of presynaptic receptors: present knowledge and future perspectives”, Trends in Pharmac. Sci., 18/3, 95-99, 1997.
12. L. Linner, L, Arborelius, G. G. Nomikos, T. H. Svensson; “Locus Coeruleus neuronal activity and NA availability in the frontal cortex of rats chronically treated with imipramine: effect of α₂-adrenoceptor blockage”, Biol. Psychiatry, 46, 766-774, 1999.
13. M Maes, I. Libbrecht, F. van Hunsel, H. Y. Meltzer; “Pindolol and mianserin augment the antidepressant activity of fluoxetine in hospitalized major depressed patients, including those with treatment resistance”, Journal of Clinical Psychopharmacology, 19, 177-182, 1999.
14. M. Marcus, K. Jardemark, P Hertel, T. H. Svensson; “Combined α₂- and D_{2/3} receptor blockage enhances cortical glutamatergic transmission and reverses cognitive impairment in the rat”, Int. J. Neuropsychopharmacol. 8, 315-327, 2005
15. O. T. Osman, M. V. Rudorfer, W. Z. Potter; “Idazoxan: a selective α₂ antagonist and effective sustained antidepressant in two Bipolar Depressed patients”, Arch. Gen. Psychiatry, 46, 958-959, 1989.
16. A. Ozerdem, M. E. Shmidt, W. Z. Potter; “Chronic lithium administration enhances noradrenergic responses to intravenous administration of the a2 antagonist idazoxan in healthy volunteers”, Journal of Clinical Psychopharmacology, 24, 150-154, 2004.
17. J. P. Reneric, M. Bouvard, L. Stinus; “Idazoxan and 8-OH-DPAT modity the behavioural effects induced by either NA, or 5-HT, or dual NA/5-HT reuptake inhibition in the rat forced swimming test”, Neuropsychopharmacology, 24, 379-390, 2001.
18. M. E. Schmidt, R. C. Risinger, R. L. Hauger and W. Z. Potter; “Responses to α₂-adrenoceptor blockade by idazoxan in healthy male and female volunteers”, Psychoneuroendocrinology, 22, 177-188, 1997.
19. K. Starke; “Presynaptic autoreceptors in the third decade: focus on α₂-adrenoceptors”, J. of Neurochemistry, 78, 685-693, 2001.
20. T. H. Svensson, B. S. Bunney G. K. Aghajanian, “Inhibition of both noradrenergic and serotonergic neurons in brain by the alpha-adrenergic agonist clonidine”. Brain Research. 92:291-306, 1975
21. T. H., Svensson, T. Usdin, “Feedback inhibition of brain noradrenaline neurons by tricyclic antidepressants: alpha-receptor mediation”. Science. 202:1089-91, 1978
22. T. H. Svensson, “Brain noradrenaline and the mechanisms of action of antidepressant drugs”. Acta Psychiatrica Scandinavica 101(402):18-27, 2000.
23. T. H. Svensson “Mode of action of atypical antipsychotic drugs: Focus on α₂ adrenoceptors”. Eur. J. Neuropsychopharmacology 14, S139, 2004
24. P. Weikop, J. Kehr, J. Scheel-Kruger; “The role of α₁- and α₂-adrenoceptors on venlafaxine-induced elevation of extracellular serotonin, noradrenaline and dopamine levels in the rat prefrontal cortex and hippocampus”, J. of Psychopharmacology, 18, 395-403, 2004.

1. Use of 2-(1,4-benzodioxan-2-yl)-2-imidazoline (Idazoxan) or an active enantiomer thereof for the manufacture of a medicament for the treatment of a patient receiving therapy with selective serotonin uptake inhibitor(s).

2. Use according to claim 1, wherein the patient is suffering from severe depression, anxiety-depression, PTSD (Post Traumatic Stress Disorder), or ADHD (Attention Deficit Hyperactive Disorder).

3. Use according to claim 2, wherein the patient exhibits poor response to monotherapy with the selective serotonin uptake inhibitor(s).

4. Use according to claim 1, wherein the medicament comprises both Idazoxan or an active enantiomer thereof and selective serotonin uptake inhibitor(s).

5. Use according to claim 1, wherein the selective serotonin uptake inhibitor(s) is (are) selected from the group consisting of Fluoxetine, Citalopram, Paroxetine, Sertraline and Fluvoxamine.

6. Use according to claim 1, wherein the medicament is in the form of a slow-release preparation.

7. A method of treating a patient receiving therapy with selective serotonin uptake inhibitor(s) comprising administration to said patient of an amount of 2-(1,4-benzodioxan-2-yl)-2-imidazoline (Idazoxan) or an active enantiomer thereof which is a psychologically effective amount together with the amount of administered selective serotonin uptake inhibitor(s).

8. The method according to claim 7, wherein the patient is suffering from severe depression, anxiety-depression, PTSD (Post Traumatic Stress Disorder), or ADHD (Attention Deficit Hyperactive Disorder).

9. The method according to claim 8, wherein the patient exhibits poor response to monotherapy with the selective serotonin uptake inhibitor(s).

10. The method according to claim 7, wherein the selective serotonin uptake inhibitor(s) and the Idazoxan or an active enantiomer thereof are administered in one pharmaceutical preparation.

11. The method according to claim 7, wherein the selective serotonin uptake inhibitor(s) is (are) selected from the group consisting of Fluoxetine, Citalopram, Paroxetine, Sertraline and Fluvoxamine.

12. The method according to claim 7, wherein the Idazoxan or an active enantiomer thereof is in the form of a slow-release preparation.

13. The method according to claim 10, wherein the pharmaceutical preparation is a slow-release preparation.

14. Use according to claim 2, wherein the medicament comprises both Idazoxan or an active enantiomer thereof and selective serotonin uptake inhibitor(s).

15. Use according to claim 2, wherein the selective serotonin uptake inhibitor(s) is (are) selected from the group consisting of Fluoxetine, Citalopram, Paroxetine, Sertraline and Fluvoxamine.

16. Use according to claim 2, wherein the medicament is in the form of a slow-release preparation.
